(19) United States
(12) Patent Application Publication
(10) Pub. No.: US 2026/0248539 A1
(43) Pub. Date: Aug. 27, 2026

(54) EXPANDABLE SI JOINT FIXATION DEVICE WITH DEPLOYABLE ANCHORS

(71) Applicant: Vy Spine LLC, Bountiful, UT (US)

(72) Inventors: Bret Michael Berry, Tallahasse, FL (US); James Yue, Hartford, CT (US)

(21) Appl. No.: 19/550,038

(22) Filed: Feb. 25, 2026

Related U.S. Application Data
(60) Provisional application No. 63/763,199, filed on Feb. 25, 2025.

(51) Int. Cl. A61B 17/70; A61B 17/68

(52) U.S. Cl. CPC A61B 17/7065; A61B 17/683; A61B 17/7064

(57) ABSTRACT

An expandable sacroiliac (SI) joint fixation device includes a housing translationally coupled to a distal pivot body along a longitudinal axis. At least one endplate is pivotally coupled to the distal pivot body and configured to pivot outwardly from a closed position to an expanded position upon translation of the housing toward the distal pivot body. At least one deployable anchor is movably coupled to the distal pivot body and configured to move from a retracted position within the endplate to a deployed position extending outwardly through the endplate and into bone adjacent to the SI joint. An actuation mechanism, including a screw drive, is configured to simultaneously cause the endplates to expand and the anchors to deploy, providing both joint distraction and enhanced fixation in a single actuation step. The device is inserted in a compact configuration through a posterior approach and expanded in situ.

### CROSS-REFERENCE TO RELATED APPLICATIONS

This application claims the benefit of U.S. Provisional Patent Application No. 63/763,199, filed Feb. 25, 2025, entitled “EXPANDABLE SI JOINT FIXATION DEVICE WITH DEPLOYABLE ANCHORS,” the entire disclosure of which is incorporated herein by reference in its entirety.

### FIELD OF THE INVENTION

The present invention relates generally to orthopedic medical devices, and more particularly to an expandable fixation device for stabilizing the sacroiliac (SI) joint, the device including deployable anchors for enhanced fixation into bone adjacent to the SI joint.

### BACKGROUND OF THE INVENTION

Sacroiliac (SI) joint dysfunction is a significant source of lower back and pelvic pain, affecting a substantial portion of the population. The SI joint connects the sacrum to the ilium on each side of the pelvis and is responsible for transferring loads between the upper body and the lower extremities. Dysfunction of the SI joint can result from degenerative changes, trauma, pregnancy-related ligamentous laxity, or other pathological conditions that compromise the structural integrity and stability of the joint.

Conservative treatments for SI joint dysfunction include physical therapy, medication management, and injection-based therapies. When conservative measures fail to provide adequate relief, surgical intervention may be necessary. SI joint fusion is a surgical procedure aimed at stabilizing the joint by promoting bony fusion between the sacrum and the ilium, thereby eliminating the abnormal motion that causes pain.

Various surgical techniques and devices have been developed for SI joint fusion. Traditional approaches have utilized dowels or wedges made from cadaveric bone, which are inserted from a medial-posterior approach and are generally hammered or threaded into the space between the sacrum and ilium. However, dowels, being generally cylindrical in shape, may not provide optimal fixation due to their round cross-section, which can allow rotation or migration within the joint space. Other devices have employed threaded screws, pins, or allograft implants to achieve fixation.

While some existing fixation devices incorporate anchoring features to aid in fixation, many current devices suffer from inadequate purchase in the bone surrounding the SI joint, which can lead to implant loosening, migration, or subsidence over time. Additionally, many current devices do not provide controlled distraction of the joint during implantation, which can be beneficial for promoting fusion by increasing the surface area available for bony ingrowth and allowing introduction of bone graft material.

Accordingly, there is a need in the art for an improved SI joint fixation device that provides enhanced anchoring and stability, controlled joint distraction, and reliable long-term fixation. The present invention addresses these and other needs.

### SUMMARY OF THE INVENTION

The present invention provides an expandable sacroiliac (SI) joint fixation device with deployable anchors that addresses the shortcomings of existing devices. The device is configured for insertion across the SI joint from a posterior approach and provides both controlled distraction of the joint and enhanced fixation through deployable anchors that engage the sacrum and ilium.

In one aspect, the invention provides an expandable SI joint fixation device comprising a housing that is translationally attached to a distal pivot body. At least one pair of endplates is pivotally coupled to the distal pivot body, and a screw drive mechanism is configured to cause the housing to translate toward the distal pivot body, thereby causing the endplates to pivot outwardly from the device body and distract the SI joint. At least one deployable anchor is movably coupled to the distal pivot body and is configured to move from a retracted position within the endplates to a deployed position in which the anchor extends outwardly through an opening in the endplate and into bone adjacent to the SI joint. An actuation mechanism, including at least the screw drive and a linkage assembly, is configured to simultaneously cause the endplates to expand and the anchors to deploy when the screw drive is actuated.

In another aspect, the invention provides a method of fixating a sacroiliac joint comprising inserting an expandable fixation device across the SI joint in an unexpanded configuration, the fixation device including a housing, a distal pivot body, at least one pair of endplates, and at least one deployable anchor in a retracted position. The method further comprises actuating a screw drive to cause the housing to translate toward the distal pivot body, thereby simultaneously expanding the endplates outwardly to distract the SI joint and deploying the at least one anchor outwardly through the endplates and into bone on at least one side of the SI joint.

In various embodiments, the device may include a pair of anchors arranged to engage bone on both sides of the SI joint, namely the sacrum and the ilium, providing bilateral fixation. The anchors may include cutting points configured to penetrate bone tissue. The endplates may include anchor openings through which the anchors extend when deployed.

The simultaneous expansion and anchor deployment mechanism provides a significant advantage over existing devices, as it enables both joint distraction and enhanced fixation in a single actuation step, reducing surgical complexity and operative time. The expandable design also allows the device to be inserted in a compact, unexpanded configuration through a minimally invasive approach and then expanded in situ to fill the joint space and anchor into surrounding bone.

### BRIEF DESCRIPTION OF THE DRAWINGS

The foregoing and other features and advantages of the present invention will become apparent from the following detailed description when read in conjunction with the accompanying drawings, in which:

FIG. 1 is a perspective view of an expandable SI joint fixation device in an expanded configuration, in accordance with an embodiment of the present invention;

FIG. 2 is a perspective view of the expandable SI joint fixation device of FIG. 1 in an unexpanded configuration;

FIG. 3 is a lateral view of the expandable SI joint fixation device of FIG. 1 in the unexpanded configuration;

FIG. 4 is a lateral view of the expandable SI joint fixation device of FIG. 1 in the expanded configuration;

FIG. 5 is a posterior view of the expandable SI joint fixation device of FIG. 1 in the expanded configuration;

FIG. 6 is a perspective view of the expandable SI joint fixation device of FIG. 1 in the expanded configuration with endplates removed to show internal components;

FIG. 7 is a perspective view of a housing component of the expandable SI joint fixation device of FIG. 1; and

FIG. 8 is a perspective view of a distal pivot body component of the expandable SI joint fixation device of FIG. 1.

### DETAILED DESCRIPTION OF THE INVENTION

The following detailed description is provided to enable any person skilled in the art to make and use the present invention. Various modifications to the embodiments described herein will be readily apparent to those skilled in the art, and the generic principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

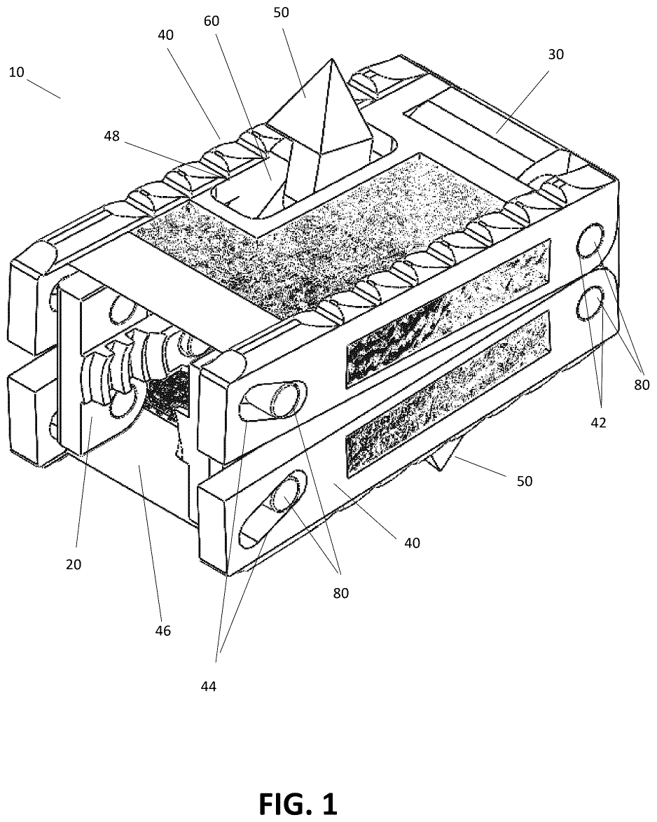

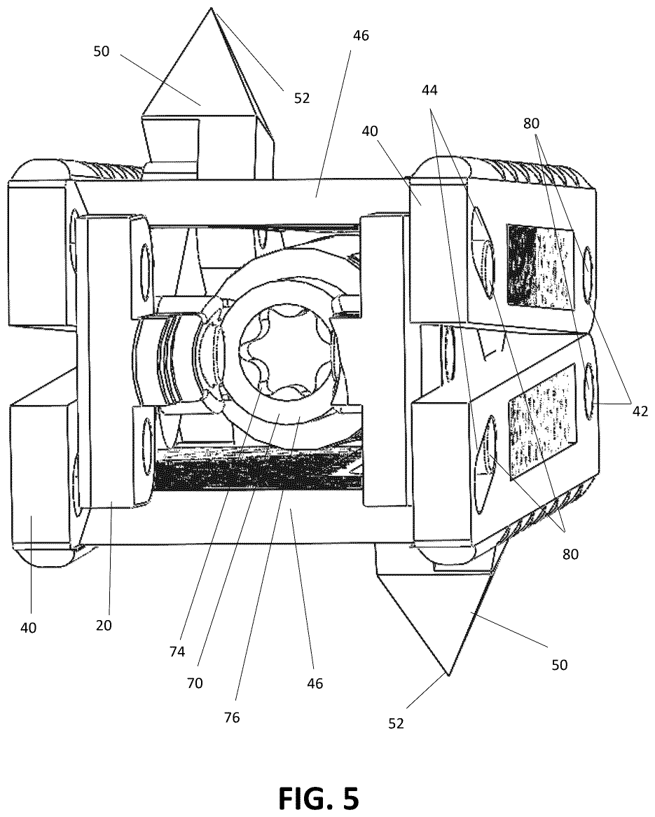

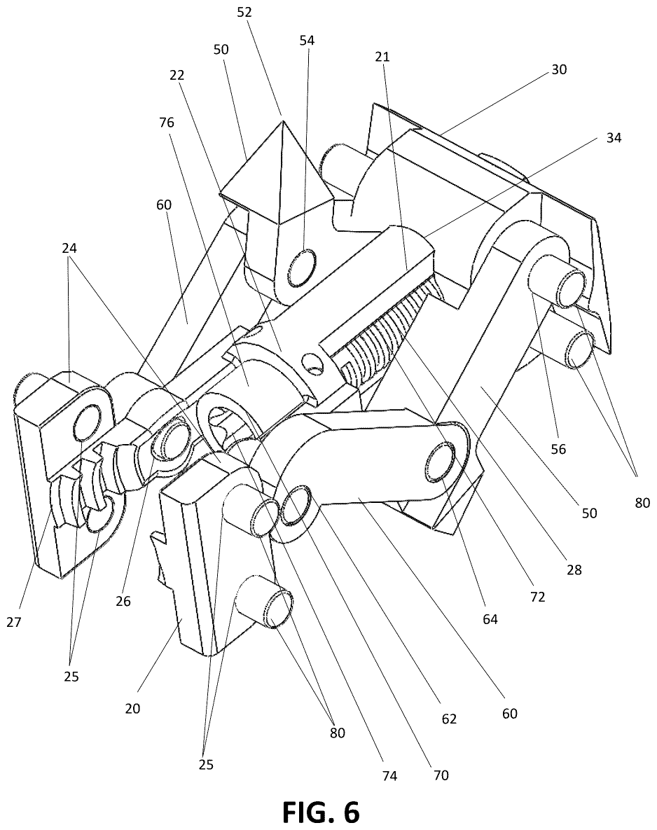

Referring now to the drawings, wherein like reference numerals refer to like components throughout the several views, FIGS. 1-8 illustrate an expandable sacroiliac (SI) joint fixation device 10 (hereinafter “implant 10” or “device 10”) in accordance with embodiments of the present invention.

Referring to FIGS. 1-6, the implant 10 comprises an assembly including a housing 20 that is translationally attached to a distal pivot body 30. The housing 20 includes rails 23 that are slidably received within a rail guide slot 34 of the distal pivot body 30. This rail and slot configuration permits the housing 20 to translate linearly with respect to the distal pivot body 30 along a longitudinal axis of the implant 10 while restricting relative rotation between the housing 20 and the distal pivot body 30.

A screw drive 70 is housed within a screw housing 21 formed in the housing 20. The screw drive 70 includes a head 76 that bears against a screw retainer 22 of the housing 20. The screw drive 70 further includes threads 72 that mate with drive threads 36 formed in the distal pivot body 30. The screw drive 70 includes a drive feature 74 at its proximal end that is configured to receive a complementary driver tool (not shown). By rotating the screw drive 70 via the drive feature 74, the threads 72 engage the drive threads 36, causing the housing 20 to be drawn toward the distal pivot body 30 in a controlled manner. In at least some embodiments, the actuation mechanism includes the screw drive 70 and the linkage assembly. In at least some embodiments, the linkage assembly includes, but is not limited to, one or more of (i) the anchor arms 60, (ii) the anchors 50, and (iii) the pins 80 received by the anchor arms 60 and the anchors 50. In at least some embodiments, the anchor arm 60 and the anchor 50 are connectable to provide a mechanical linkage or pivoting arm assembly.

Referring to FIGS. 1, 2, 4, and 6, a pair of endplates 40 are pivotally attached to the distal pivot body 30 by pins 80 that extend through endplate pivot pin holes 42 at a distal end of each endplate 40 and seat into corresponding body pivot pin holes 32 of the distal pivot body 30. The endplates 40 are configured to rotate about the pins 80 relative to the distal pivot body 30.

Each endplate 40 includes ramps 46 at its proximal end that ride along corresponding ramps 24 formed at a proximal portion of the housing 20. Additionally, pins 80 extend from slot pin holes 25 of the housing 20 into pin slots 44 located at a proximal portion of each endplate 40. The pin slots 44 serve to constrain the motion of the endplates 40 and prevent the endplates 40 from prematurely expanding prior to intentional actuation.

As the housing 20 is drawn toward the distal pivot body 30 by actuation of the screw drive 70, the ramps 46 of the endplates 40 ride against the ramps 24 of the housing 20, causing the endplates 40 to (i) pivot outwardly about the pins 80 at the distal end and (ii) slide outwardly on the pins 80 at the proximal end. This outward movement of the endplates 40 expands the overall profile of the implant 10, thereby distracting the sacroiliac joint and creating space for bony ingrowth and fusion.

Referring to FIGS. 4, 5, and 6, at least one anchor 50 is pivotally attached to the distal pivot body 30 via a pin 80 extending through a pivot pin hole 56 of the anchor 50 and into the body pivot pin holes 32 of the distal pivot body 30. The anchor 50 is configured to rotate about this pin 80. An arm pivot pin hole 54 of the anchor 50 is connected to an anchor arm 60 via a pin 80 extending through an anchor pivot pin hole 64 of the anchor arm 60. At the opposite end of the anchor arm 60 from the anchor pivot pin hole 64, the anchor arm 60 is pivotally connected to the housing 20 via a pin 80 extending through a housing pivot pin hole 62 and into anchor arm pin holes 26 on the housing 20.

The anchor 50 is generally L-shaped, with the pivot pin hole 56 at one end, the arm pivot pin hole 54 at the elbow of the L-shape, and a cutting point 52 at the opposite end from the pivot pin hole 56. This L-shaped geometry, in conjunction with the linkage or pivoting arm assembly formed by at least one or more of the anchor arm 60 and the anchor 50, converts the linear translation of the housing 20 into rotational deployment of the anchor 50.

As the housing 20 is drawn toward the distal pivot body 30 during actuation, the anchor arm 60 causes the anchor 50 to rotate about the pivot pins 80 that connect the distal pivot body 30 and the anchor arm 60 to the anchor 50, forcing the cutting point 52 of the anchor 50 outwardly through an anchor opening 48 formed in the endplate 40. The cutting point 52 is thereby driven into the bone of the sacrum or ilium adjacent to the SI joint. This deployment of the anchor 50 occurs simultaneously with the expansion of the endplates 40, as both motions are driven by the same linear translation of the housing 20 toward the distal pivot body 30.

In a preferred embodiment, the implant 10 includes a pair of anchors 50, with one anchor 50 on each side of the device that corresponds to one of the endplates 40. The anchors 50 are configured to deploy through the respective endplate 40 and engage bone on both sides of the SI joint, with, one anchor 50 engaging the sacrum and the other anchor 50 engaging the ilium. This bilateral anchoring provides enhanced fixation and resistance to migration or loosening.

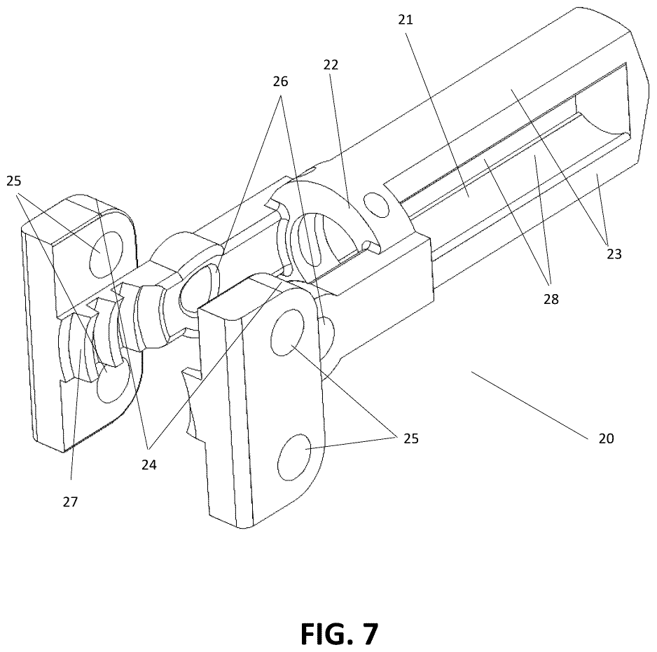

Referring to FIG. 7, the housing 20 is shown in isolation. The housing 20 includes the screw housing 21 for receiving the screw drive 70, screw thread openings 28 to permit the threads 72 of the screw drive 70 to engage with the drive threads 36 of the distal pivot body 30, the screw retainer 22 for retaining the head 76 of the screw drive 70, the rails 23 for slidable engagement with the rail guide slot 34 of the distal pivot body 30, the ramps 24 for engaging the endplate ramps 46, the slot pin holes 25 for receiving pins 80 that engage with the pin slots 44 of the endplates 40 to enable the endplate 40 to slide and expand relative to the housing, the anchor arm pin holes 26 that receive pins 80 for pivotally connecting the anchor arms 60 to the housing 20 via the housing pivot pin holes 62, and inserter threads 27 for attachment of a surgical inserter instrument (not shown).

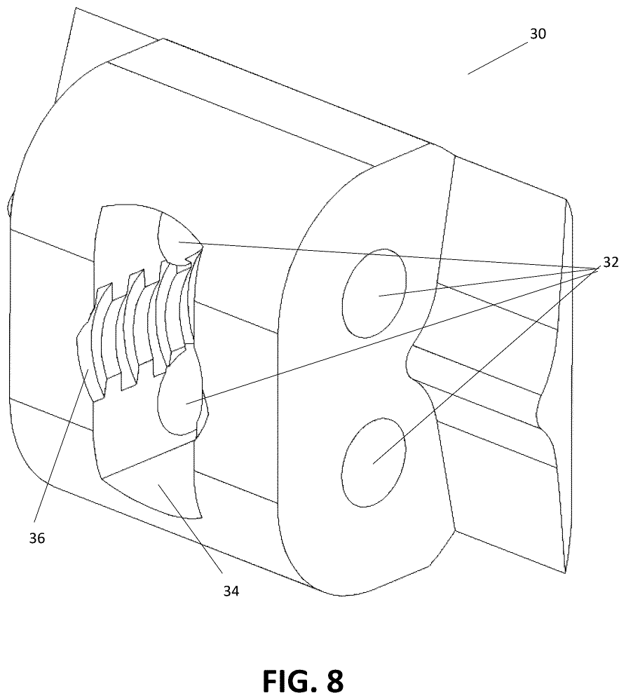

Referring to FIG. 8, the distal pivot body 30 is shown in isolation. The distal pivot body 30 includes the body pivot pin holes 32 that receive pins 80 for pivotally connecting the endplate pivot pin holes 42 of the endplates 40 and the pivot pin hole 56 of the anchors 50, the rail guide slot 34 for slidably receiving the rails 23 of the housing 20, and the drive threads 36 for engaging the threads 72 of the screw drive 70.

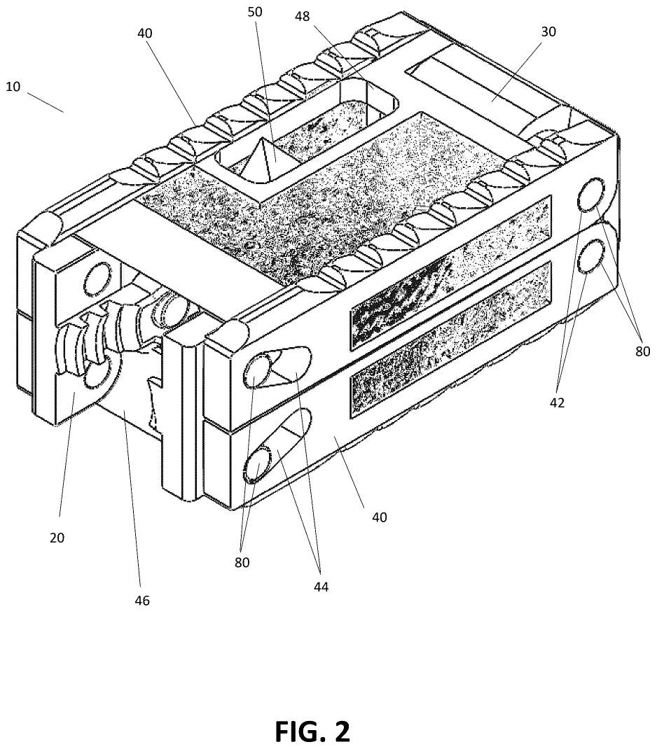

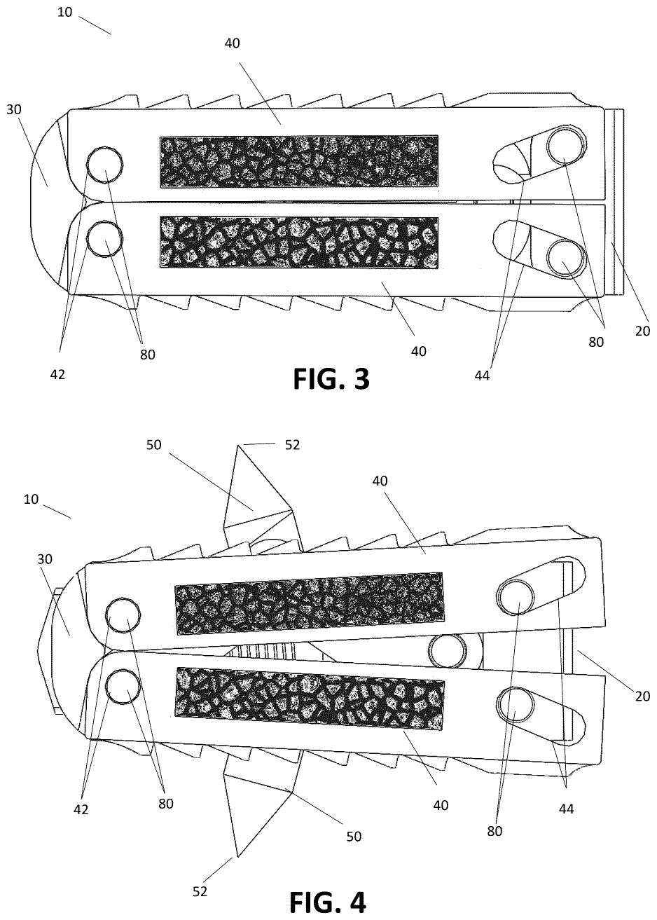

A method of use for the implant 10 will now be described. The implant 10 is inserted into the sacroiliac joint in an unexpanded configuration (FIGS. 2-3) using a surgical inserter (not shown) attached to the inserter threads 27 on the housing 20. In this unexpanded configuration, the endplates 40 are in a closed, low-profile position, and the anchors 50 are retracted within the endplates 40, providing a compact profile suitable for insertion through a minimally invasive posterior approach. In a preferred embodiment, when in the retracted position within the endplates 40, the anchors 50 may be at or below the outermost surface of any portion of the endplates 40. In some embodiments, when in the retracted position within the endplates 40, the anchors 50 may be completely concealed within the endplates.

Once the implant 10 is appropriately positioned within the joint space between the sacrum and ilium, a driver tool (not shown) is used to engage the drive feature 74 of the screw drive 70 and rotate the screw drive 70. This rotation of the screw drive 70 causes the housing 20 to be drawn toward the distal pivot body 30 via the threaded engagement between the threads 72 and the drive threads 36.

As the housing 20 translates toward the distal pivot body 30, two simultaneous actions occur: first, the endplates 40 (i) pivot outwardly about the pivot pins 80 in the endplate pivot pin holes 42 and (ii) slide outwardly on the pins 80 in the pin slots 44, expanding the implant 10 and distracting the sacroiliac joint; second, the anchors 50 are deployed outwardly through the anchor openings 48 in the endplates 40, with the cutting points 52 of the anchors 50 being driven into the sacral and iliac bone on either side of the joint. The simultaneous expansion and anchor deployment is achieved through a single actuation mechanism, simplifying the surgical procedure and reducing operative time.

The expanded implant 10 with deployed anchors 50 provides a stable construct that resists migration, loosening, and subsidence, while the distracted joint space facilitates bony ingrowth and eventual fusion of the sacroiliac joint. The implant 10 may be fabricated from biocompatible materials suitable for orthopedic implants, such as titanium, titanium alloys, cobalt-chromium alloys, stainless steel, polyetheretherketone (PEEK), or other suitable materials. The endplates 40 and/or other bone-contacting surfaces of the implant 10 may be provided with textured, roughened, or porous coatings to promote bone ingrowth and osseointegration.

While the foregoing description has been directed to specific embodiments of the present invention, it will be apparent to those of ordinary skill in the art that various modifications and variations can be made to the described embodiments without departing from the spirit and scope of the invention. All such modifications and variations are intended to fall within the scope of the appended claims.

What is claimed is:

1. An expandable joint fixation device, comprising:
a housing having a longitudinal axis;
a distal pivot body configured to receive the housing, wherein the distal pivot body translates linearly along the housing with respect the longitudinal axis of the housing;
an endplate pivotally coupled to the distal pivot body and configured to pivot from a closed position to an expanded position outwardly away from the housing upon translation of the housing toward the distal pivot body;
a deployable anchor coupled to the distal pivot body, wherein the deployable anchor is movable between a retracted position within the expandable joint fixation device and a deployed position where the deployable anchor extends beyond the endplate and into bone adjacent to the expandable joint fixation device; and
an actuation mechanism configured to move the housing relative to the distal pivot body to simultaneously pivot the endplate to the expanded position and move the anchor to the deployed.

2. The device of claim 1, wherein the actuation mechanism includes a screw drive rotatably retained within the housing and having threads that mate with drive threads formed in the distal pivot body, whereby rotation of the screw drive in a first direction causes the housing to translate toward the distal pivot body.

3. The device of claim 2, wherein the housing is formed with a screw thread opening through which the threads of the screw are exposed to mate with the drive threads of the distal pivot body.

4. The device of claim 1, wherein the housing includes rails that are slidably received within a rail guide slot of the distal pivot body, the rails and rail guide slot configured to permit linear translation while restricting relative rotation between the housing and the distal pivot body.

5. The device of claim 1, wherein the endplate includes ramps at a first end that engage corresponding ramps on the housing, such that translation of the housing toward the distal pivot body causes the endplate to pivot toward the expanded position.

6. The device of claim 1, wherein the deployable anchor is L-shaped and includes a pivot pin hole at a first end, an arm pivot pin hole at an elbow, and a cutting point at a second end.

7. The device of claim 1, further comprising an anchor arm pivotally connecting the deployable anchor to the housing, wherein translation of the housing toward the distal pivot body causes the anchor arm to rotate the deployable anchor from the retracted position to the deployed position.

8. The device of claim 1, wherein the endplate includes an anchor opening through which a portion of the deployable anchor extends when the deployable anchor is in the deployed position.

9. The device of claim 1, further comprising a second endplate pivotally coupled to the distal pivot body on an opposite side of the device from the endplate, wherein the second endplate configured to pivot outwardly away from the housing upon actuation of the actuation mechanism.

10. The device of claim 9, further comprising a second deployable anchor configured to deploy through the second endplate.

11. The device of claim 10, wherein the deployable anchor and the second deployable anchor are configured to engage with different bones across a joint.

12. The device of claim 1, wherein the deployable anchor is retracted below an outer surface of the endplate when the endplate is in the closed position.

13. The device of claim 1, further comprising at least one pin constraining the endplate to prevent premature expansion of the endplate prior to actuation of the actuation mechanism.

14. The device of claim 1, wherein the housing includes inserter threads configured for attachment of a surgical inserter instrument.

15. An expandable joint fixation device, comprising:
a housing having a longitudinal axis;
a distal pivot body configured to receive the housing, wherein the distal pivot body translates linearly along the housing with respect the longitudinal axis of the housing;
a pair of endplates pivotally coupled to housing and the distal pivot body, wherein each endplate of the pair of endplates is configured to pivot from a closed position to an expanded position outwardly away from the housing upon translation of the housing toward the distal pivot body; and
an actuation mechanism comprising,
a linkage assembly comprising,
a first deployable anchor and a second deployable anchor each coupled to the distal pivot body, wherein the first deployable anchor and the second deployable anchor are each movable between a retracted position within the expandable joint fixation device and a deployed position where a portion of the first deployable anchor and a portion of the second deployable anchor extend beyond the endplate and into bone adjacent to the expandable joint fixation device,
a first anchor arm connected between the first deployable anchor and a first side of the housing, wherein the first deployable anchor and the first anchor arm form a first pivoting arm assembly, and
a second anchor arm connected between the second deployable anchor and a second side of the housing, wherein the second deployable anchor and the second anchor arm form a second pivoting arm assembly, and
a screw drive rotatably retained within the housing and between the first pivoting arm assembly and the second pivoting arm assembly, wherein the screw drive is configured to simultaneously pivot the pair of endplates to the expanded position and move the first anchor and the second anchor to the deployed position.

16. A method of fusion for a sacroiliac (SI) joint, comprising:
inserting an expandable fixation device across the SI joint in an unexpanded configuration, the fixation device including a housing, a distal pivot body translationally coupled to the housing, at least one endplate pivotally coupled to the distal pivot body, and at least one deployable anchor in a retracted position;
actuating an actuation mechanism to cause the housing to translate toward the distal pivot body; and
simultaneously expanding the at least one endplate outwardly to distract the SI joint and deploying the at least one deployable anchor outwardly through the at least one endplate and into bone adjacent to the SI joint.

17. The method of claim 16, wherein actuating the actuation mechanism comprises rotating a screw drive housed within the housing, the screw drive threadedly engaging the distal pivot body.

18. The method of claim 16, wherein the fixation device is inserted through a posterior approach.

19. The method of claim 16, wherein deploying the at least one deployable anchor comprises deploying a pair of anchors on opposite sides of the fixation device to engage bone on both sides of the SI joint.

20. The method of claim 16, wherein the at least one deployable anchor includes a cutting point that is driven into at least one of the sacrum and the ilium upon deployment.
